(19) United States
(12) Patent Application Publication
(10) Pub. No.: US 2025/0019401 A1
(43) Pub. Date: Jan. 16, 2025

(54) ANTIMICROBIAL PEPTIDE BOLESPLEENIN OF BOLEOPHTHALMUS PECTINIROSTRIS AND APPLICATION THEREOF

(71) Applicant: Xiamen University, Xiamen (CN)

(72) Inventors: Kejian WANG, Xiamen City (CN); Yuqi BAI, Xiamen City (CN); Fangyi CHEN, Xiamen City (CN); Hui PENG, Xiamen City (CN); Huiyun CHEN, Xiamen City (CN)

(21) Appl. No.: 18/661,017

(22) Filed: May 10, 2024

(30) Foreign Application Priority Data
Jul. 13, 2023 (CN) 202310859711.3

(51) Int. Cl. C07K 7/08; A61K 38/00; A61P 31/04; A61P 31/10

(52) U.S. Cl. CPC C07K 7/08; A61P 31/04; A61P 31/10; A61K 38/00

(57) ABSTRACT

An antimicrobial peptide Bolespleenin of Boleophthalmus pectinirostris and application thereof, and an amino acid sequence of the antimicrobial polypeptide Bolespleenin is shown in SEQ ID NO.01.

### RELATED APPLICATIONS

This application claims priority to Chinese patent application 202310859711.3, filed on Jul. 13, 2023. Chinese patent application 202310859711.3 is incorporated herein by reference.

### REFERENCE TO AN ELECTRONIC SEQUENCE LISTING

The contents of the electronic sequence listing (SequenceListing.xml; Size: 1,765 bytes; and Date of Creation: May 10, 2024) is herein incorporated by reference.

### FIELD OF THE DISCLOSURE

The present disclosure relates to the technical field of marine molecular biotechnology, and specifically relates to an antimicrobial peptide Bolespleenin of Boleophthalmus pectinirostris and an application thereof.

### BACKGROUND OF THE DISCLOSURE

It is reported that China is currently the largest producer and consumer of antibiotics in the world and is also one of the countries with the most serious drug resistant bacteria. The large amount of unregulated and irrational use of antibiotics has caused a series of problems, such as increased bacterial resistance, suppressed animal immunity, affected human health, and even damaged ecological environment. Therefore, the development of new and efficient anti-bacterial drugs and the search for effective antibiotic alternatives have become urgent problems to be solved.

Antimicrobial peptides (AMPs) are a kind of small-molecule anti-microbial peptides and are widely distributed in animals and plants. AMPs are the first line of defense against infections of various pathogenic microorganisms and are an important part of the innate immune system. A main disease resistance mechanism of AMPs is to act on cell membranes of pathogenic microorganisms to enable the pathogenic microorganisms to have difficulty in producing resistance, so that a generation of drug resistance problems is avoided. AMPs have a broad spectrum of anti-bacterial activity, anti-fungal activity, anti-viral activity, anti-parasitic activity, and other activities. In addition, AMPs have multiple roles, such as acting as immunomodulators, signaling molecules, anti-tumor molecules, etc. Therefore, AMPs are very attractive alternatives to traditional antibiotics and important candidates for the development of new antimicrobial drugs, and AMPs also have considerable prospects in transformation applications.

### BRIEF SUMMARY OF THE DISCLOSURE

An objective of the present disclosure is to provide an antimicrobial peptide Bolespleenin of Boleophthalmus pectinirostris and an application thereof.

A technical solution of the present disclosure is as follows:

An antimicrobial peptide Bolespleenin of Boleophthalmus pectinirostris, an amino acid sequence of the antimicrobial polypeptide Bolespleenin is shown in SEQ ID NO.01.

A method for preparing an anti-bacterial composition using the antimicrobial peptide Bolespleenin of Boleophthalmus pectinirostris.

In a preferred embodiment, the antibacterial composition has an inhibitory or killing effect on at least one of Staphylococcus aureus, Enterococcus faecium, Enterococcus faecalis, Acinetobacter baumannii, Escherichia coli, or Pseudomonas aeruginosa.

An anti-bacterial composition, an active ingredient of the antibacterial composition comprises the antimicrobial peptide Bolespleenin.

A method for preparing an antifungal composition using the antimicrobial peptide Bolespleenin.

In a preferred embodiment, the antifungal composition has an inhibitory or killing effect on at least one of Cryptococcus neoformans, Fusarium oxysporum, Aspergillus flavus, or Fusarium solani.

An antifungal composition, an active ingredient of the antifungal composition comprises the antimicrobial peptide Bolespleenin.

A method for preparing an aquatic feed additive using the antimicrobial peptide Bolespleenin.

An aquatic feed additive, an active ingredient of the aquatic feed additive comprises the antimicrobial peptide Bolespleenin.

In a preferred embodiment, an effective ingredient of the aquatic feed additive is the antimicrobial peptide Bolespleenin.

The technical solution has the following advantages:

1. The antimicrobial peptide Bolespleenin of Boleophthalmus pectinirostris of the present disclosure consists of 14 amino acids with a molecular formula of C₈₃H₁₄₃N₃₃O₁₆ and a molecular weight of 1859.26 Daltons. The antimicrobial peptide Bolespleenin contains 5 amino acid residues positively charged, and the isoelectric point of the antimicrobial peptide Bolespleenin is predicted to be 12.18 based on charges of the 5 amino acid residues. The average hydrophilicity coefficient of the antimicrobial peptide Bolespleenin is −0.779, and the antimicrobial peptide Bolespleenin is highly water-soluble, and is a cationic peptide positively charged.

2. The present disclosure has a significant antibacterial effect on Gram-positive bacteria, Gram-negative bacteria, and some fungi. Further, the antimicrobial peptide Bolespleenin has no cytotoxic effect on normal zebrafish embryonic cells and normal mammalian cells, such as human embryonic kidney 293T cells (HEK 293T cells) at a concentration of no more than 48 μMol/L (μM).

3. The present disclosure has good antimicrobial effect, broad antimicrobial spectrum, and a rapid bactericidal rate. The antimicrobial peptide Bolespleenin is derived from marine teleost fishes, can be used in aquatic feed additives, can also be developed as anti-bacterial compositions, anti-fungal compositions and so on, and has a broad application prospect.

### BRIEF DESCRIPTION OF THE DRAWING

FIGS. 1A and 1B show bactericidal kinetics of the antimicrobial peptide Bolespleenin of Boleophthalmus pectinirostris against Staphylococcus aureus and Acinetobacter baumannii in Embodiment 3 of the present disclosure. FIG. 1A shows Staphylococcus aureu suspension mixed with 6 μMol/L (μM) of the antimicrobial peptide Bolespleenin, and FIG. 1B shows Acinetobacter baumannii suspension mixed with 6 μM of the antimicrobial peptide Bolespleenin. In FIGS. 1A and 1B, the X-axes are time (minutes), and the Y-axes are bactericidal rates (%).

FIGS. 2A and 2B show thermal stability of antibacterial activity of the antimicrobial peptide Bolespleenin of Boleophthalmus pectinirostris against Acinetobacter baumannii in Embodiment 4 of the present disclosure. In FIG. 2A, the X-axis is temperature (° C.), and the Y-axis is a number of colonies (CFU/mL). In FIG. 2B, the X-axis is time (minutes), and the Y-axis is the number of colonies (CFU/mL).

FIGS. 3A, 3B, 3C, 3D, 3E, 3F, 3G, and 3H show inhibition of the antimicrobial peptide Bolespleenin of Boleophthalmus pectinirostris against Fusarium oxysporum spore germination in Embodiment 5 of the present disclosure. Final concentrations of the antimicrobial peptide Bolespleenin are respectively 0 μM, 1.5 μM, 3 μM, 6 μM, 12 μM, 24 μM, 48 μM, and 96 μM.

FIGS. 4A, 4B, 4C, 4D, 4E, 4F, 4G, and 4H show inhibition of the antimicrobial peptide Bolespleenin of Boleophthalmus pectinirostris against Aspergillus flavus spore germination in Embodiment 5 of the present disclosure. Final concentrations of the antimicrobial peptide Bolespleenin are respectively 0 μM, 1.5 μM, 3 μM, 6 μM, 12 μM, 24 μM, 48 μM, and 96 μM.

FIGS. 5A, 5B, 5C, 5D, 5E, 5F, 5G, and 5H show morphological changes of bacteria and fungi after treatment with the antimicrobial peptide Bolespleenin of Boleophthalmus pectinirostris in Embodiment 6 of the present disclosure under scanning electron microscope (SEM) observation. FIG. 5A shows an Acinetobacter baumannii control, FIG. 5B shows a treatment of Acinetobacter baumannii with the antimicrobial peptide Bolespleenin having a final concentration of 3 μM, FIG. 5C shows a Staphylococcus aureus control, FIG. 5D shows a treatment of Staphylococcus aureus with the antimicrobial peptide Bolespleenin having a final concentration of 3 μM, FIG. 5E shows an Aspergillus flavus control, FIG. 5F shows the treatment of Aspergillus flavus with the antimicrobial peptide Bolespleenin having a final concentration of 48 μM, FIG. 5G shows a Fusarium oxysporum control, and FIG. 5H shows the treatment of Fusarium oxysporum with the antimicrobial peptide Bolespleenin having a final concentration of 6 μM.

FIGS. 6A and 6B show graphs of cytotoxicity tests of the antimicrobial peptide Bolespleenin of Boleophthalmus pectinirostris in Embodiment 7 of the present disclosure using a 3-(4,5-dimethylthiazol-2-yl)-5-(3-carboxymethoxyphenyl)-2-(4-sulfophenyl)-2H-tetrazolium-phenazine methosulfate (MTS-PMS) assay. FIG. 6A is 293T cells, and FIG. 6B is ZF4 cells. In FIGS. 6A and 6B, the X-axes are peptide concentrations (μM) of the antimicrobial peptide Bolespleenin, and the Y-axes are cell proliferation rates (%).

### DETAILED DESCRIPTION OF THE EMBODIMENTS

The technical solution of the present disclosure will be further described in combination with the accompanying drawings and embodiments.

### Embodiment 1 Preparation of an Antimicrobial Peptide Bolespleenin of Boleophthalmus pectinirostris (Mudskipper)

The amino acid sequence of the antimicrobial peptide Bolespleenin of Boleophthalmus pectinirostris is: Leu-Ile-Gly-Leu-Tyr-Leu-Leu-His-Arg-Arg-Arg-Arg-Arg-His (as shown in SEQ ID NO.01).

An existing solid phase chemical synthesis method can be used to obtain the antibacterial peptide Bolespleenin of Boleophthalmus pectinirostris with a purity of no less than 95%. The antibacterial peptide Bolespleenin of Boleophthalmus pectinirostris in Embodiments 1-7 is synthesized by Genscript (Nanjing, China) by the solid phase synthesis method, and analysis information, such as peptide molecular weight and high performance liquid chromatography (HPLC), is provided.

The physicochemical parameters of the antimicrobial peptide Bolespleenin are shown in Table 1.

TABLE 1
The physicochemical parameters of the antimicrobial peptide
Bolespleenin

| Physicochemical parameters | Bolespleenin |
|---|---|
| Amino acid residues | 14 |
| Molecular weight | 1859.26 Da |
| Molecular formula | C₈₃H₁₄₃N₃₃O₁₆ |
| Isoelectric point | 12.18 |
| Net charge | +5 |
| Hydrophobicity | 34.1% |
| Total average hydrophilicity | −0.779 |
| Molar extinction coefficient | 1490 |

The antibacterial peptide Bolespleenin is a cationic peptide positively charged with a small molecular weight, good stability, and highly water-soluble performance, as shown in Table 1.

### Embodiment 2: Determination of Minimum Inhibitory Concentration (MIC) and Minimum Bactericidal Concentration (MBC) of the Antimicrobial Peptide Bolespleenin of Boleophthalmus pectinirostris

Bacterial strains involved in Embodiment 2 are as follows: Staphylococcus aureus, Acinetobacter baumannii, Escherichia coli, Enterococcus faecium, Enterococcus faecalis, Pseudomonas aeruginosa, Cryptococcus neoformans, Fusarium oxysporum, Aspergillus flavus, and Fusarium solani.

The bacterial strains are purchased from the China General Microbiological Culture Collection Center (CGMCC).

A specific analysis method of this embodiment is as follows:

(1) The conserved strains are coated on a nutrient broth plate, a Yeast Extract Peptone Dextrose (YPD) plate, or a potato dextrose plate and are inverted and incubated for 1-7 days.

(2) Colonies are picked from the plates, inoculated on a slant with a corresponding medium, continually incubated for 1-7 days, and washed down from the slant with corresponding medium with 10 mMol/L (mM) of sodium phosphate buffer (pH=7.4), and concentrations of suspensions of the bacteria or suspensions of the fungi are adjusted. The suspensions of the bacteria are diluted so that final concentrations are 5×10⁵ CFU/mL. Mold spores of the suspensions of the fungi are counted under an optical microscope using a hemocyte counting plate, and concentrations of mold spores are adjusted to final concentrations of 5×10⁴ spores/mL.

(3) Powder of the synthesized antimicrobial peptide Bolespleenin is dissolved in sterilized distillation-distillation H₂O (ddH₂O), filtered through a 0.22 μm filter membrane, and concentrations of the antimicrobial peptide Bolespleenin are then diluted to 3 μM, 6 μM, 12 μM, 24 μM, 48 μM, 96 μM, and 192 μM, and placed on ice for use.

(4) A blank control group, a negative control group, and an experimental group of each of the bacteria and the fungi to be tested are set on 96-well cell culture plates, and each of the blank control group, the negative control group, and the experimental group has three parallel samples.

The blank control group a: 50 μL of the antimicrobial peptide Bolespleenin with 50 μL of the corresponding medium;

The negative control group b: 50 μL of the sterilized ddH₂O with 50 μL of each of the suspensions of the bacteria or the suspensions of the fungi; and

The experimental group c: 50 μL of the antimicrobial peptide Bolespleenin with 50 μL of each of the suspensions of the bacteria or the suspensions of the fungi.

(5) The 96-well cell culture plates are plated in an incubator at 28° C. and incubated for 1-2 days, and MIC results of the experimental group are observed. The experimental group is mixed by blowing, an appropriate amount of each of the suspensions of the bacteria or the suspensions of the fungi is sucked up, added dropwise onto the plate with the corresponding medium, and inverted and incubated at an appreciate temperature for 1-2 days, and MBC results are observed.

The MIC and MBC results of the antimicrobial peptide Bolespleenin are shown in Table 2:

TABLE 2
Antimicrobial activity of the antimicrobial peptide Bolespleenin

| | | MIC | MBC |
|---|---|---|---|
| Microorganism | CGMC. NO. | (a-b) | (a-b) |
| Gram-positive bacterium | | | |
| Staphylococcus aureus | 1.2465 | <1.5 | 1.5-3 |
| Enterococcus faecium | 1.131 | 1.5-3 | 1.5-3 |
| Enterococcus faecalis | 1.2135 | 3-6 | 3-6 |
| Gram-negative bacterium | | | |
| Acinetobacter baumannii | 1.6769 | 1.5-3 | 1.5-3 |
| Escherichia coli | 1.2385 | 3-6 | 6-12 |
| Pseudomonas aeruginosa | 1.2421 | <1.5 | 1.5-3 |
| Fungi | | | |
| Cryptococcus neoformans | 2.1563 | <1.5 | <1.5 |
| Fusarium oxysporum | 3.6785 | 3-6 | 3-6 |
| Aspergillus flavus | 3.4410 | 6-12 | 24-48 |
| Fusarium solani | 3.584 | 3-6 | 3-6 |

Note:
a-b represent minimum inhibitory concentration (MIC) (μM) and minimum bacterial concentration (MBC) (μM).
a: The highest concentration of the antimicrobial peptide Bolespleenin that induces visible growth of microorganisms.
b: The lowest concentration of the antimicrobial peptide Bolespleenin that does not induce visible growth of microorganisms.

### Embodiment 3: A Bactericidal Kinetic Curve of the Antimicrobial Peptide Bolespleenin of Boleophthalmus pectinirostris

Staphylococcus aureus and Acinetobacter baumannii are selected to determine bactericidal kinetics of the antimicrobial peptide Bolespleenin of Boleophthalmus pectinirostris.

A specific method is similar to the antimicrobial activity test described in Embodiment 2. The antimicrobial peptide Bolespleenin is adjusted to 1×MBC (for Staphylococcus aureus, a final concentration of the antimicrobial peptide Bolespleenin is 3 μM; for Acinetobacter baumannii, a final concentration of the antimicrobial peptide Bolespleenin is 3 μM). After co-incubation with Staphylococcus aureus or Acinetobacter baumannii for a period of time, the blank control group, the negative control group, and the experimental group are taken from the 96-well cell culture plates and mixed to even. 6 μL of each of the suspensions of the bacteria is sucked up, diluted using 600 μL of Dulbecco's phosphate-buffered saline (DPBS), and mixed to even to obtain a dilution, 40 μL of the dilution is then sucked up, coated onto a nutrient broth plate, and then inverted and incubated at 37° C. for 1-2 days. A number of monoclones of each of the bacteria is recorded, and a bactericidal index is obtained by calculation.

The bactericidal index is a ratio of a number of clones in the experimental group and a number of clones in the negative control group after co-incubation for a preset time, and the ratio is represented by a percentage (see FIGS. 1A and 1B).

As shown in FIGS. 1A and 1B, the antimicrobial peptide Bolespleenin can kill no less than 95% of Staphylococcus aureus at the final concentration of 3 μM after 30 minutes, and the antimicrobial peptide Bolespleenin can kill no less than 95% of Acinetobacter baumannii at the final concentration of 3 μM after 60 minutes.

### Embodiment 4: Thermal Stability of Antimicrobial Activity of the Antimicrobial Peptide Bolespleenin of Boleophthalmus pectinirostris

Staphylococcus aureus is selected to determine thermal stability of antimicrobial activity of the antimicrobial peptide Bolespleenin of Boleophthalmus pectinirostris.

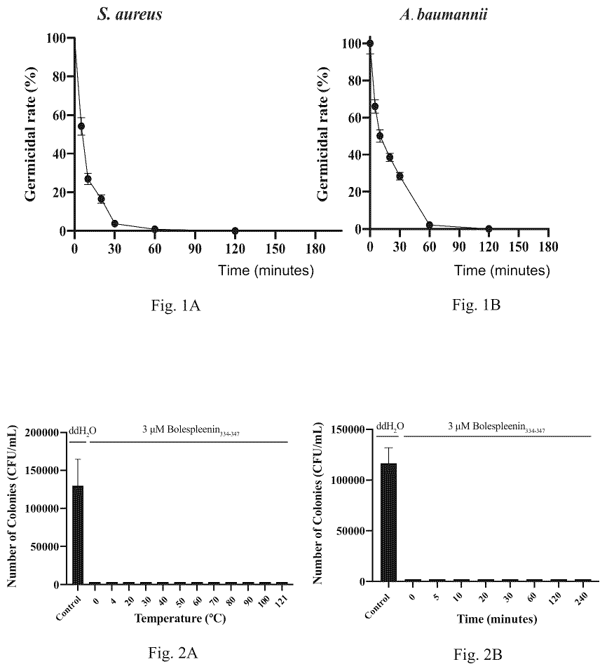

A specific method is similar to the antimicrobial activity test described in Embodiment 2. A final concentration of the antimicrobial peptide Bolespleenin is adjusted to 1×MBC (for Acinetobacter baumannii, the final concentration of the antimicrobial peptide Bolespleenin is 3 μM). After a solution of the antimicrobial peptide Bolespleenin is reserved at different temperatures for 30 minutes, cooled by placing on ice for 10 minutes, then co-incubated with Staphylococcus aureus (in the negative control group, sterilized ddH₂O is co-incubated with Staphylococcus aureus) for 4 hours, coated on a plate, incubated in an incubator at 37° C. for 10 hours (as shown in FIG. 2A), and then counted; The antimicrobial peptide Bolespleenin is placed in 100° C. boiling water for water bath for different durations and then placed on ice. The antimicrobial peptide Bolespleenin or the sterilized ddH₂O is respectively co-incubated with Staphylococcus aureus for 4 hours, coated on a plate, incubated at 37° C. for 10 hours in an incubator (as shown in FIG. 2B), and counted. As shown in FIGS. 2A and 2B, changes in temperature gradients or sustained high temperatures (e.g., 100° C.) did not affect the activity of the antimicrobial peptide Bolespleenin.

### Embodiment 5: Microscopic Observations of Spore Germination of Mold after Treatment with the Antimicrobial Peptide Bolespleenin of Boleophthalmus pectinirostris

Fusarium oxysporum and Aspergillus flavus are selected to observe effects of the antimicrobial peptide Bolespleenin of Boleophthalmus pectinirostris on the spore germination of the mold.

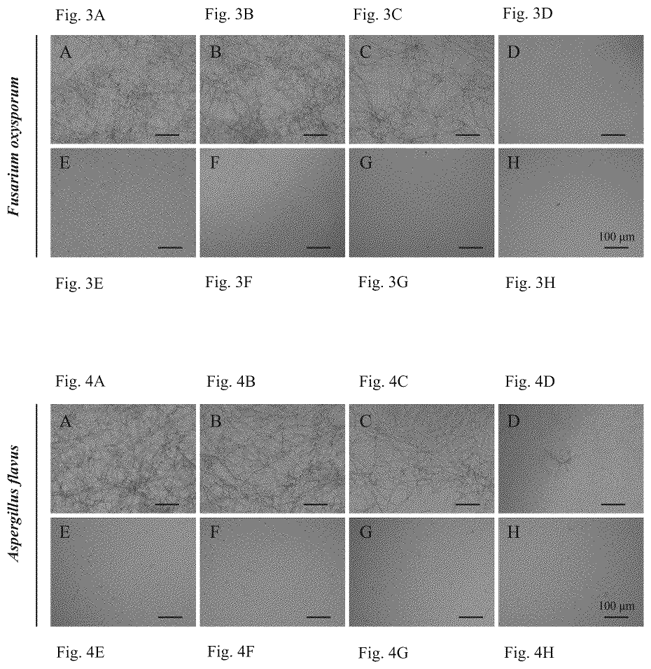

A specific method is similar to the antimicrobial activity test described in Embodiment 2. Concentrations of the antimicrobial peptide Bolespleenin are adjusted to 6 μM, 12 μM, 24 μM, 48 μM, 96 μM, and 192 μM, and placed on ice for use. A final concentration of spores each of the molds is adjusted to 5×10⁴ spores/mL. Each of the antimicrobial peptides Bolespleenin with equal volumes and various concentrations and the spores of each of molds are mixed in 96-well cell culture plates, and then incubated in an incubator at 28° C. for 24 hours using stationary cultivation, and the spore germination of the mold is observed under the optical microscope. As shown in FIGS. 3A, 3B, 3C, 3D, 3E, 3F, 3G, and 3H and FIGS. 4A, 4B, 4C, 4D, 4E, 4F, 4G, and 4H, the spore germination is observed under the optical microscope, and the antimicrobial peptide Bolespleenin has a significant inhibitory effect on the spore germination of Fusarium oxysporum and Aspergillus flavus respectively under a final concentration of 6 μM and 12 μM.

### Embodiment 6: Scanning Electron Microscope (SEM) Observation of Bacteria and Fungi after Treatment with the Antimicrobial Peptide Bolespleenin of Boleophthalmus pectinirostris

Acinetobacter baumannii, Staphylococcus aureus, Aspergillus flavus, and Fusarium oxysporum are selected as strains to be tested, and samples for scanning electron microscopy are prepared according to the following steps:

(1) Suspension solutions of Acinetobacter baumannii and Staphylococcus aureus (OD600=0.4) and spore suspensions of Aspergillus flavus and Fusarium oxysporum (5×10⁶ spores/mL) are prepared as the method described in Embodiment 2 and placed on ice for use.

(2) The synthesized antimicrobial peptide Bolespleenin is dissolved with sterilized purified water, concentrations of the antimicrobial peptide Bolespleenin are adjusted to 6 μM, 12 μM, and 96 μM to obtain protein solutions, and the protein solutions are placed on ice for use.

(3) Each of the suspension solutions and each of the protein solutions with the same volume are mixed and incubated at an appropriate temperature for an appropriate time (the appropriate temperature and the appropriate time are selected according to MIC and sterilization kinetic criteria). Final concentrations of the antimicrobial peptide Bolespleenin are therefore 3 μM, 6 μM, and 48 μM.

(4) Glutaraldehyde fixative solution with the same volume is added, fixed at 4° C. for 2 hours, and then centrifuged at 6000 g for 5 minutes. A supernatant is removed, and precipitate is resuspended in 1 mL of phosphate-buffered saline (PBS) and centrifuged at 6000 g for 10 minutes. The step is repeated.

(5) 10 μL of PBS is added to prepare a strain suspension with a high concentration. The strain suspension with the high concentration is added dropwise on a slide, placed on ice, left to stand for 30 minutes, and excessive liquid is adsorbed by filter paper to be removed.

(6) After the strain suspension with the high concentration is immersed in the PBS for 15 minutes and is dehydrated in step concentrations of 30%, 50%, 70%, 80%, 90%, 95%, 100%, and 100% (volume/volume (v/v)) of ethanol, dehydration in each of the step concentrations is 15 minutes.

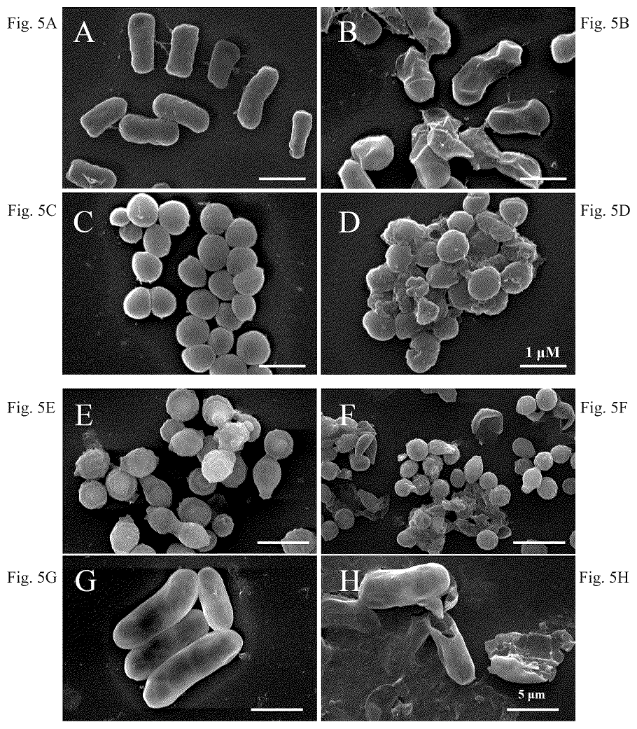

(7) The samples are dried using a critical point drying method. After a gold spray, the samples are observed and photographed by a scanning electron microscope (SEM) (as shown in FIGS. 5A, 5B, 5C, 5D, 5E, 5F, 5G, and 5H).

As shown in FIGS. 5A, 5B, 5C, 5D, 5E, 5F, 5G, and 5H, the strains in the negative control group have normal morphologies, intact structures, and smooth surfaces. The strains treated with the antimicrobial peptide Bolespleenin have morphologies that change significantly and wrinkles, holes, or even ruptures of membrane surfaces appear, which resulted in contents leaking out.

### Embodiment 7: Determination of Cytotoxicity of the Antimicrobial Peptide Bolespleenin of Boleophthalmus pectinirostris

Human renal epithelial cells (293T) and zebrafish embryo fibroblasts (ZF4) are selected to determine the cytotoxicity of the antimicrobial peptide Bolespleenin of Boleophthalmus pectinirostris.

(1) Well grown human renal epithelial cells and zebrafish embryo fibroblasts are harvested, concentrations of human renal epithelial cells and zebrafish embryo fibroblasts are adjusted to 1×10⁵ cells/mL, the cells are blown to even to obtain cell suspensions, 100 μL of each of the cell suspensions is added into 96-well cell culture plates per well and incubated in an incubator at an appropriate temperature until no less than 80% of the cells are attached to the wall.

(2) Mediums of the human renal epithelial cells and the zebrafish embryo fibroblasts are sucked out, and mediums containing different concentrations of the antimicrobial peptide Bolespleenin are added and incubated in an incubator at an appropriate temperature for 24 hours.

(3) 20 μL of a 3-(4,5-dimethylthiazol-2-yl)-5-(3-carboxymethoxyphenyl)-2-(4-sulfophenyl)-2H-tetrazolium-phenazine methosulfate (MTS-PMS) solution is added and then incubated away from light for 2 hours, and the cytotoxicity of the antimicrobial peptide Bolespleenin is evaluated by measuring an OD492 value using a microplate reader.

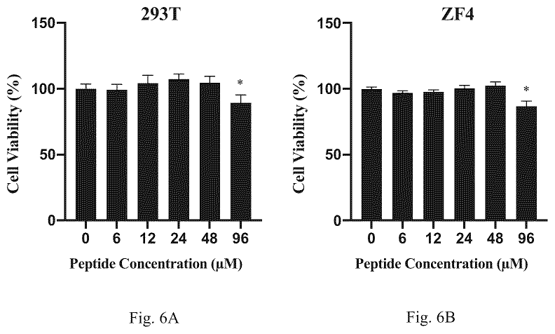

As shown in FIGS. 6A and 6B, the antimicrobial peptide Bolespleenin is merely cytotoxic to human renal epithelial cells and zebrafish embryo fibroblasts at a concentration up to 96 μM, and no cytotoxicity of the antimicrobial peptide Bolespleenin is observed at the concentration of no more than 48 μM.

The aforementioned embodiments are merely some embodiments of the present disclosure, and the scope of the disclosure is not limited thereto. Thus, it is intended that the present disclosure cover any modifications and variations provided they are made without departing from the appended claims and the specification of the present disclosure.

What is claimed is:

1. An antimicrobial peptide Bolespleenin of Boleophthalmus pectinirostris, wherein:
an amino acid sequence of the antimicrobial polypeptide Bolespleenin is shown in SEQ ID NO.01;
the amino acid sequence of the antimicrobial polypeptide Bolespleenin is the sequence shown in the SEQ ID NO.01 with at least one amino acid residue replaced, deleted, or inserted; or
the amino acid sequence of the antimicrobial polypeptide Bolespleenin has more than 80% homology with the sequence shown in the SEQ ID NO.01.

2. A method for preparing an anti-bacterial composition using the antimicrobial peptide Bolespleenin of Boleophthalmus pectinirostris according to claim 1.

3. The method according to claim 2, wherein the anti-bacterial composition has an inhibitory or killing effect on at least one of Staphylococcus aureus, Enterococcus faecium, Enterococcus faecalis, Acinetobacter baumannii, Escherichia coli, or Pseudomonas aeruginosa.

4. An anti-bacterial composition, wherein an active ingredient of the anti-bacterial composition comprises the antimicrobial peptide Bolespleenin according to claim 1.

5. A method for preparing an antifungal composition using the antimicrobial peptide Bolespleenin according to claim 1.

6. The method according to claim 5, wherein the antifungal composition has an inhibitory or killing effect on at least one of Cryptococcus neoformans, Fusarium oxysporum, Aspergillus flavus, or Fusarium solani.

7. An antifungal composition, wherein an active ingredient of the antifungal composition comprises the antimicrobial peptide Bolespleenin according to claim 1.

8. A method for preparing an aquatic feed additive using the antimicrobial peptide Bolespleenin according to claim 1.

9. An aquatic feed additive, wherein an active ingredient of the aquatic feed additive comprises the antimicrobial peptide Bolespleenin according to claim 1.

10. The aquatic feed additive according to claim 9, wherein an effective ingredient of the aquatic feed additive is the antimicrobial peptide Bolespleenin.


SEQUENCE LISTING (1 sequence)

SEQ ID NO: 1 - protein, 14 aa, Boleophthalmus pectinirostris
LIGLYLLHRRRRRH